(19) United States
(12) Patent Application Publication
(10) Pub. No.: US 2026/0325274 A1
(43) Pub. Date: Oct. 22, 2026

(54) METHOD FOR MANUFACTURING DENTAL RESIN BLOCK AND DENTAL RESIN BLOCK

(71) Applicant: INNOMEDI CO., LTD., Nonsan-si (KR)

(72) Inventor: Yun Chul LEE, Daejeon (KR)

(73) Assignee: INNOMEDI CO., LTD., Nonsan-si (KR)

(21) Appl. No.: 19/695,112

(22) Filed: Jun. 2, 2026

Related U.S. Application Data
(63) Continuation of application No. PCT/KR2025/005868, filed on Apr. 30, 2025.

(30) Foreign Application Priority Data
Jun. 24, 2024 (KR) 10-2024-0082240

(51) Int. Cl. A61C 13/00; A61K 6/71; A61K 6/884

(52) U.S. Cl. CPC A61C 13/0006; A61K 6/71; A61K 6/884

(57) ABSTRACT

A method for manufacturing a dental resin block is provided. The method comprises preparing a block composition that comprises a base resin, an inorganic filler, and a monomer; mixing the block composition; press-molding the mixed block composition; and curing the press-molded block composition under a first wavelength range of 395 nm to 550 nm and a second wavelength range of 200 nm to 393 nm. A dental resin block manufactured by the method is also provided.

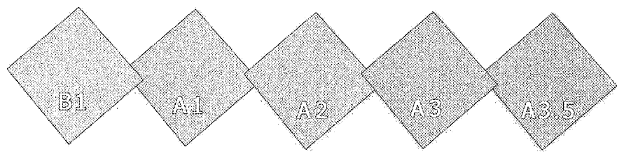

### CROSS REFERENCE TO RELATED APPLICATIONS

This application is a Continuation of PCT/KR2025/005868 filed Apr. 30, 2025 which claims priority to Korean Patent Application No. 10-2024-0082240 filed Jun. 24, 2024, the entire disclosures of which are incorporated herein by reference.

### TECHNICAL FIELD

The present disclosure relates to a method for manufacturing a dental resin block and a dental resin block.

### BACKGROUND ART

Currently, digital restoration based on computer-aided design and computer-aided manufacturing (CAD/CAM) technology has become a significant direction of development in dental restoration. Various dental restorations may be digitally fabricated.

Ceramic materials are among the most widely used materials in CAD/CAM technology due to their excellent mechanical properties, optical properties, and biocompatibility. However, ceramic materials tend to be brittle and may be subject to spontaneous cracking, which may cause substantial wear of milling burs. The relatively high hardness of ceramic materials may also cause severe wear of natural teeth.

Resin materials, on the other hand, offer relatively low cost, excellent toughness, ease of machining and cutting, shorter restoration cycles, and reduced no wear on natural teeth, and accordingly occupy a significant share of CAD/CAM milling applications. However, resin materials may have relatively low strength, relatively low wear resistance, and a relatively short service life, which may limit their use as permanent restorative materials.

Resin-ceramic restorative materials may combine certain strength characteristics of ceramic materials with the toughness of resin materials. However, two issues may be encountered. (1) Resin-ceramic restorative materials may inherit some biocompatibility characteristics of ceramic materials, while higher strength associated with the ceramic component may cause wear of natural teeth and may render the material susceptible to brittle fracture. Although the mechanical properties may be improved as compared with purely resin materials, the actual mechanical performance of resin-ceramic composite materials in dental restoration may not fully reach a desirable level. (2) Resin-ceramic restorative materials may exhibit a relatively high water absorption, which may degrade their mechanical properties, may induce microleakage, may reduce the thermal stability of the material, may cause precipitation of unreacted monomers, and may even cause hydrolysis of polymer segments, thereby potentially shortening the service life of the material.

### DISCLOSURE

### Technical Problem

The present disclosure provides a method for manufacturing a dental resin block and a dental resin block that may address one or more of the above-described considerations.

### Technical Solution

An exemplary embodiment of the present disclosure provides a method for manufacturing a dental resin block, the method including: preparing a block composition including a base resin, an inorganic filler, and a monomer; mixing the block composition; press-molding the mixed block composition; and curing the press-molded block composition under a first wavelength range of from about 395 nm to about 550 nm and a second wavelength range of from about 200 nm to about 393 nm.

Another exemplary embodiment of the present disclosure provides a dental resin block manufactured by the above-described manufacturing method.

### Advantageous Effects

According to an exemplary embodiment of the present disclosure, the method for manufacturing a dental resin block may facilitate color realization, and may thereby provide a customized color suitable for matching the natural color of a patient's tooth.

### DESCRIPTION OF DRAWINGS

The FIGURE is a photograph showing colors of resin blocks according to Examples 1 to 5.

### BEST MODE

An exemplary embodiment of the present disclosure provides a method for manufacturing a dental resin block, the method including: preparing a block composition including a base resin, an inorganic filler, and a monomer; mixing the block composition; press-molding the mixed block composition; and curing the press-molded block composition under a first wavelength range of from about 395 nm to about 550 nm and a second wavelength range of from about 200 nm to about 393 nm.

Conventional dental resin blocks manufactured by related-art methods may exhibit relatively low mechanical rigidity, or may readily undergo discoloration.

The present inventors have developed a method for manufacturing a dental resin block that may address such considerations.

According to an exemplary embodiment of the present disclosure, in the method for manufacturing a dental resin block, the block composition serving as a raw material may comprise all of a base resin, an inorganic filler, and a monomer, such that the mechanical rigidity attributable to the inorganic filler may be improved, and the mechanical rigidity associated with the curing properties of the monomer may be enhanced. In addition, because the method comprises curing the press-molded block composition under a first wavelength range of from about 395 nm to about 550 nm and a second wavelength range of from about 200 nm to about 393 nm, uniform curing properties may be maintained even when various materials are included, and discoloration and similar considerations may be addressed.

The method for manufacturing a dental resin block according to an exemplary embodiment of the present disclosure comprises preparing a block composition including a base resin, an inorganic filler, and a monomer. The base resin, the inorganic filler, and the monomer may each be prepared through a separate manufacturing process, or commercially available materials may be used.

The method for manufacturing a dental resin block according to an exemplary embodiment of the present disclosure comprises mixing the block composition. This step is a step of dispersing the respective components together. The manufacturing method may comprise, prior to the step of mixing the block composition (hereinafter referred to as the main mixing step), a step of separately dispersing each of the base resin, the inorganic filler, and the monomer (hereinafter referred to as the pre-dispersion step). By performing the pre-dispersion step in advance, the respective materials may be uniformly mixed in the main mixing step, and the mixing efficiency may be enhanced.

The method for manufacturing a dental resin block according to an exemplary embodiment of the present disclosure comprises press-molding the mixed block composition. This step is a step of molding the block composition into a desired shape.

The method for manufacturing a dental resin block according to an exemplary embodiment of the present disclosure comprises curing the press-molded block composition under a first wavelength range of from about 395 nm to about 550 nm and a second wavelength range of from about 200 nm to about 393 nm. This step may be a step of inducing a crosslinking reaction of the block composition by electron beam in the first and second wavelength ranges.

In an exemplary embodiment of the present disclosure, the step of curing the press-molded block composition is performed under a first wavelength range of from about 395 nm to about 550 nm and a second wavelength range of from about 200 nm to about 393 nm. Under these wavelength conditions, the curing properties of the monomer included in the composition may be improved, and the quality of the resulting product may thereby be enhanced. Performing the curing under the first wavelength range and the second wavelength range may comprise simultaneously irradiating with the first wavelength range and the second wavelength range using light sources having different wavelength ranges. In this case, the light source may comprise a halogen lamp, a high-pressure mercury lamp, or the like.

In an exemplary embodiment of the present disclosure, the first wavelength range may be from about 397 nm to about 500 nm, and in some embodiments may be from about 400 nm to about 420 nm. Under such conditions, monomers having a photosensitive wavelength range longer than the ultraviolet range may be sufficiently cured.

In an exemplary embodiment of the present disclosure, the second wavelength range may be from about 220 nm to about 390 nm, and in some embodiments may be from about 250 nm to about 387 nm. Under such conditions, monomers having a photosensitive wavelength range corresponding to the ultraviolet range may be sufficiently cured.

In an exemplary embodiment of the present disclosure, a difference between the first wavelength range and the second wavelength range may be from about 1 nm to about 300 nm. In some embodiments, the difference may be from about 5 nm to about 150 nm, or from about 5 nm to about 50 nm. Under such conditions, the entirety of the monomers may exhibit relatively uniform curing properties.

In an exemplary embodiment of the present disclosure, the step of curing the press-molded block composition may be performed for a duration of from about 5 hours to about 24 hours. In some embodiments, the duration may be from about 8 hours to about 15 hours, or from about 10 hours to about 13 hours. Under such conditions, the curing may be sufficient such that the quality of the finished product may be enhanced and damage to the product may be reduced.

In an exemplary embodiment of the present disclosure, the step of curing the press-molded block composition may be performed while the block composition is disposed in a mold.

In an exemplary embodiment of the present disclosure, the step of preparing the block composition including the base resin, the inorganic filler, and the monomer may comprise separately dispersing each of the base resin, the inorganic filler, and the monomer in independent systems. In this case, the respective components may be uniformly mixed in the subsequent step of mixing the block composition. The independent systems mean that, while each material is being dispersed, that material does not contact the other materials. For example, the base resin, the inorganic filler, and the monomer may be dispersed in respective separate vessels.

In an exemplary embodiment of the present disclosure, the step of separately dispersing each of the base resin, the inorganic filler, and the monomer may be performed under conditions of from about 1,000 rpm to about 5,000 rpm for from about 1 minute to about 30 minutes. In some embodiments, the step may be performed under conditions of from about 1,300 rpm to about 3,000 rpm for from about 3 minutes to about 20 minutes, or under conditions of from about 1,700 rpm to about 2,600 rpm for from about 4 minutes to about 10 minutes. In this numerical range, each of the materials may be sufficiently dispersed.

In an exemplary embodiment of the present disclosure, the monomer may comprise a high-viscosity monomer and a low-viscosity monomer having viscosities different from each other, and the step of mixing the block composition may comprise sequentially introducing the high-viscosity monomer and the low-viscosity monomer into a mixer. Because the monomer comprises the high-viscosity monomer and the low-viscosity monomer having different viscosities, mechanical rigidity and storage stability attributable to the high-viscosity monomer may be enhanced, and processability and adhesiveness attributable to the low-viscosity monomer may be improved. The step of mixing the block composition may comprise sequentially introducing the high-viscosity monomer and the low-viscosity monomer into a mixer.

In an exemplary embodiment of the present disclosure, a time interval between introduction of the high-viscosity monomer and introduction of the low-viscosity monomer may be from about 1 second to about 10 minutes. In some embodiments, the interval may be from about 10 seconds to about 5 minutes, or from about 30 seconds to about 3 minutes. Under such conditions, the high-viscosity monomer may be favorably blended with the low-viscosity monomer, and processability may be enhanced.

In an exemplary embodiment of the present disclosure, dynamic viscosity may be measured and calculated by methods known in the art. For example, dynamic viscosity may be measured by a flow meter, and may be calculated as dynamic viscosity using Newton's equation.

In an exemplary embodiment of the present disclosure, the high-viscosity monomer may have a dynamic viscosity at about 25° C. of about 3,000 cPs or more, and the low-viscosity monomer may have a dynamic viscosity at about 25° C. of about 2,500 cPs or less. In this numerical range, the mechanical rigidity and storage stability attributable to the high-viscosity monomer may be improved, and the processability and adhesiveness attributable to the low-viscosity monomer may be further improved.

The viscosity ranges and the order of introduction of the high-viscosity monomer and the low-viscosity monomer are described above because the flow properties and curing properties of the high-viscosity monomer and the low-viscosity monomer may differ from each other, and uniform curing as well as physical properties attributable to the high-viscosity monomer and the low-viscosity monomer themselves may be further enhanced.

In an exemplary embodiment of the present disclosure, the high-viscosity monomer may have a dynamic viscosity at about 25° C. of about 3,000 cPs or more and about 30,000 cPs or less, or of about 3,500 cPs or more and about 15,000 cPs or less. In this numerical range, the mechanical rigidity and storage stability attributable to the high-viscosity monomer may be improved.

In an exemplary embodiment of the present disclosure, the low-viscosity monomer may have a dynamic viscosity at about 25° C. of about 1 cPs or more and about 2,500 cPs or less. In this numerical range, the processability and adhesiveness attributable to the low-viscosity monomer may be further improved. In an exemplary embodiment of the present disclosure, the low-viscosity monomer may comprise a first low-viscosity monomer having a dynamic viscosity at about 25° C. of about 1 cPs or more and about 500 cPs or less, and a second low-viscosity monomer having a dynamic viscosity at about 25° C. of about 600 cPs or more and about 2,500 cPs or less.

In an exemplary embodiment of the present disclosure, the first low-viscosity monomer may have a dynamic viscosity at about 25° C. of about 2 cPs or more and about 100 cPs or less, or of about 3 cPs or more and about 50 cPs or less.

In an exemplary embodiment of the present disclosure, the second low-viscosity monomer may have a dynamic viscosity at about 25° C. of about 650 cPs or more and about 1,500 cPs or less, or of about 700 cPs or more and about 1,200 cPs or less.

In an exemplary embodiment of the present disclosure, the step of mixing the block composition may be performed under mixing conditions of from about 100 rpm to about 600 rpm for from about 1 hour to about 48 hours. In some embodiments, the step may be performed under mixing conditions of from about 120 rpm to about 500 rpm for from about 6 hours to about 36 hours, or under mixing conditions of from about 150 rpm to about 390 rpm for from about 12 hours to about 30 hours. Under such conditions, the raw materials may be sufficiently mixed, occurrence of a dead zone within the mixing vessel may be reduced, and processability may be improved. The dispersion conditions (rpm, time) of the pre-dispersion step may differ from the dispersion conditions (rpm, time) of the main mixing step.

In an exemplary embodiment of the present disclosure, the step of press-molding the mixed block composition may comprise increasing temperature from a starting temperature of from about 25° C. to about 35° C. up to a final temperature of from about 100° C. to about 130° C. at a heating rate of from about 2° C./hr to about 12° C./hr. Under such conditions, the physical properties of the material may be maintained, and thermal damage to the raw materials may be reduced.

In an exemplary embodiment of the present disclosure, the starting temperature may be from about 28° C. to about 33° C., or from about 29° C. to about 31° C. Under such conditions, thermal damage to the material before the press step may be reduced.

In an exemplary embodiment of the present disclosure, the final temperature may be from about 125° C. to about 130° C., or from about 128° C. to about 130° C. Under such conditions, the material may be sufficiently molded, and thermal damage to the material may be reduced.

In an exemplary embodiment of the present disclosure, the heating rate may be from about 4° C./hr to about 11° C./hr, or from about 6° C./hr to about 10° C./hr. Under such conditions, the physical properties of the material may be maintained, and thermal damage to the raw materials may be reduced.

In an exemplary embodiment of the present disclosure, the step of press-molding the mixed block composition may be performed under a pressure of from about 5 MPa to about 50 MPa for from about 5 hours to about 48 hours. In some embodiments, the step may be performed under a pressure of from about 20 MPa to about 40 MPa for from about 8 hours to about 24 hours, or under a pressure of from about 25 MPa to about 35 MPa for from about 10 hours to about 15 hours. In this numerical range, the physical properties of the material may be maintained, and thermal damage to the raw materials may be reduced.

In an exemplary embodiment of the present disclosure, the base resin may comprise a polyaryletherketone resin. The polyaryletherketone may comprise, as repeating units, an aryl group, an ether group, and a ketone group. In this case, the polyaryletherketone may have a linear polymer structure in which aryl groups are bonded through ether groups and ketone groups. The aryl group may be a phenyl group.

In an exemplary embodiment of the present disclosure, the base resin may have a glass transition temperature of from about 120° C. to about 200° C. In some embodiments, the glass transition temperature may be from about 130° C. to about 180° C., or from about 140° C. to about 150° C. In this numerical range, resistance under high-temperature processing conditions may be improved.

In an exemplary embodiment of the present disclosure, the base resin may have a melting point of from about 300° C. to about 500° C. In some embodiments, the melting point may be from about 320° C. to about 450° C., or from about 330° C. to about 400° C. In this numerical range, resistance under high-temperature processing conditions may be improved, such that heat resistance may be enhanced.

In an exemplary embodiment of the present disclosure, the polyaryletherketone may be polyetherketone (PEK), polyetheretherketone (PEEK), polyetherketoneketone (PEKK), polyetherketoneetherketoneketone (PEKEKK), or a combination thereof. In some embodiments, the polyaryletherketone may comprise polyetheretherketone (PEEK).

In an exemplary embodiment of the present disclosure, the inorganic filler may have a average particle diameter (D50) of from about 0.1 μm to about 50 μm. In some embodiments, the average particle diameter may be from about 0.2 μm to about 30 μm, or from about 0.5 μm to about 20 μm. In this numerical range, miscibility with other materials may be improved.

In an exemplary embodiment of the present disclosure, the inorganic filler may comprise silica, aluminum silicate, alumina, calcium fluoride, strontium fluoride, calcium carbonate, kaolin, clay, mica, aluminum sulfate, calcium sulfate, barium sulfate, titanium oxide, calcium phosphate, hydroxyapatite, calcium hydroxide, strontium hydroxide, zeolite, titanium dioxide, zirconia or combinations thereof.

In an exemplary embodiment of the present disclosure, the inorganic filler may comprise an inorganic glass filler. The inorganic glass filler may have relatively low translucency, may provide enhanced aesthetic appearance, and may exhibit good miscibility with other inorganic fillers or with the base resin.

In an exemplary embodiment of the present disclosure, the inorganic glass filler may have a refractive index of from about 1.5 to about 1.6. In some embodiments, the refractive index may be from about 1.52 to about 1.58, or from about 1.54 to about 1.56. In this numerical range, the aesthetic appearance provided by the inorganic filler may be enhanced.

In an exemplary embodiment of the present disclosure, the inorganic glass filler may comprise one or more of fluorine glass, borosilicate glass, soda glass, barium glass, barium aluminosilicate glass, strontium-containing glass, zirconium-containing glass, glass ceramic, and fluoro-aluminosilicate glass. In an exemplary embodiment of the present disclosure, the monomer may comprise a methyl methacrylate-based monomer.

In an exemplary embodiment of the present disclosure, the monomer may comprise one or more of 2,2-bis-[4-(2-hydroxy-3-methacryloyloxypropoxy)phenyl]propane (bisphenol A glycerolate dimethacrylate, Bis-GMA), ethylene glycol dimethacrylate (EGDMA), triethylene glycol dimethacrylate (TEGDMA), bisphenol A ethoxylate dimethacrylate (Bis-EMA), urethane dimethacrylate (UDMA), dipentaerythritol pentaacrylate monophosphate (PENTA), 2-hydroxyethyl methacrylate (HEMA), polyalkenoic acid, biphenyl dimethacrylate (BPDM), glycerol phosphate dimethacrylate (GPDM), and combinations thereof.

In an exemplary embodiment of the present disclosure, the high-viscosity monomer may comprise 2,2-bis-[4-(2-hydroxy-3-methacryloyloxypropoxy)phenyl] propane (bisphenol A glycerolate dimethacrylate, Bis-GMA), urethane dimethacrylate (UDMA), or a combination thereof.

In an exemplary embodiment of the present disclosure, the low-viscosity monomer may comprise bisphenol A ethoxylate dimethacrylate (Bis-EMA), triethylene glycol dimethacrylate (TEGDMA), or a combination thereof.

In an exemplary embodiment of the present disclosure, the amount of the monomer may be from about 100 parts by weight to about 1,000 parts by weight, based on 100 parts by weight of the base resin. In some embodiments, the amount of the monomer may be from about 300 parts by weight to about 800 parts by weight, or from about 400 parts by weight to about 600 parts by weight, based on 100 parts by weight of the base resin. In this numerical range, miscibility between the monomer and the base resin may be improved, and curing efficiency may be enhanced.

In an exemplary embodiment of the present disclosure, the amount of the inorganic filler may be from about 1,000 parts by weight to about 2,000 parts by weight, based on 100 parts by weight of the base resin. In some embodiments, the amount of the inorganic filler may be from about 1,200 parts by weight to about 1,900 parts by weight, or from about 1,400 parts by weight to about 1,800 parts by weight, based on 100 parts by weight of the base resin. In this numerical range, miscibility between the monomer and the base resin may be improved, and curing efficiency may be enhanced.

In an exemplary embodiment of the present disclosure, the inorganic filler may comprise the inorganic glass filler, and the amount of the inorganic glass filler may be from about 80 parts by weight to about 98 parts by weight, based on 100 parts by weight of the total inorganic filler. In some embodiments, the amount of the inorganic glass filler may be from about 85 parts by weight to about 96 parts by weight, or from about 90 parts by weight to about 95 parts by weight, based on 100 parts by weight of the total inorganic filler. In this numerical range, the aesthetic appearance attributable to the inorganic glass filler may be favorably maintained.

In an exemplary embodiment of the present disclosure, the block composition may comprise a photoinitiator, a polymerization retarder, a pigment, or a combination thereof.

In an exemplary embodiment of the present disclosure, the photoinitiator may comprise one or more of 1-hydroxy-cyclohexyl-phenyl-ketone, diphenyl(2,4,6-trimethylbenzoyl)phosphine oxide (TPO), Irgacure 819, Irgacure 2959, Omnirad 500, thioxanthone (TX), thioxanthone derivatives, benzophenone (BPO), benzophenone derivatives, and combinations thereof.

In an exemplary embodiment of the present disclosure, the amount of the photoinitiator may be from about 5 parts by weight to about 30 parts by weight, based on 100 parts by weight of the base resin. In some embodiments, the amount may be from about 10 parts by weight to about 28 parts by weight, or from about 15 parts by weight to about 25 parts by weight. In this numerical range, photo-curing rate may be improved.

In an exemplary embodiment of the present disclosure, the polymerization retarder may comprise butylated hydroxytoluene (BHT), tribenzylamine (TBA), or a combination thereof.

In an exemplary embodiment of the present disclosure, the pigment may comprise a red pigment, a yellow pigment, a black pigment, or a combination thereof.

In an exemplary embodiment of the present disclosure, the red pigment may comprise red iron oxide.

In an exemplary embodiment of the present disclosure, the yellow pigment may comprise yellow iron oxide, yellow bismuth, yellow vanadium-zirconium, yellow cerium-praseodymium, or a combination thereof.

In an exemplary embodiment of the present disclosure, the black pigment may comprise black iron oxide.

In an exemplary embodiment of the present disclosure, the amount of the pigment may be from about 0.01 part by weight to about 5 parts by weight, based on 100 parts by weight of the base resin. In some embodiments, the amount may be from about 0.05 part by weight to about 3 parts by weight, or from about 0.1 part by weight to about 1 part by weight. In this numerical range, favorable coloring may be obtained.

The method for manufacturing a dental resin block according to an exemplary embodiment of the present disclosure may comprise removing the cured block composition from a mold and obtaining the cured block composition.

An exemplary embodiment of the present disclosure provides a dental resin block manufactured by the above-described manufacturing method.

In an exemplary embodiment of the present disclosure, the dental resin block may have a shape selected from the group consisting of a rectangular parallelepiped, a cube, a cylinder, and a sphere. Hereinafter, the present disclosure will be described in more detail through Examples.

### Preparation Example

The following materials A to F were prepared.

### Base Resin (A)

a1: polyetheretherketone (PEEK), CAS No.: 29658-26-2

### Inorganic Filler (B)

b1: barium aluminosilicate (BaAl₂O₄), D50: 0.5-20 μm, CAS No.: 12004-04-5
b2: silica (silicon dioxide, SiO₂), D50: 5-20 μm, CAS No.: 68611-44-9
b3: titanium dioxide (TiO₂), CAS No.: 13463-67-7

### Monomer (C)

c1: bisphenol A glycerolate dimethacrylate (Bis-GMA), pH: 6.2-7.9, viscosity (@ 25° C.): 4,000-8,000 cPs, CAS No.: 1565-94-2
c2: bisphenol A ethoxylate dimethacrylate (Bis-EMA), pH: 6.3-7.5, viscosity (@ 25° C.): 700-1,200 cPs, CAS No.: 64401-02-1
c3: triethylene glycol dimethacrylate (TEGDMA), viscosity (@ 25° C.): 5-15 cPs, CAS No.: 109-16-0
c4: urethane dimethacrylate (UDMA), viscosity (@ 25° C.): 5,900~11,900 cPs, CAS No.: 934705-15-4

### Photoinitiator (D)

d1: benzoyl peroxide (BPO), CAS No.: 94-36-0
d2: diphenyl(2,4,6-trimethylbenzoyl)phosphine oxide (TPO), CAS No.: 75980-60-8

### Polymerization Retarder (E)

e1: butylated hydroxytoluene (BHT), CAS No.: 128-37-0

### Pigment (F)

f1: red iron oxide (IOR), CAS No.: 1314-13-2
f2: yellow iron oxide (IOY), CAS No.: 15201-61-3
f3: black iron oxide (IOB), CAS No.: 1317-61-9

### Example 1

The base resin, the inorganic filler, and the monomer were respectively prepared in the compositional ratios shown in Table 1 below. The respective materials were thoroughly mixed at a speed of 2,500 rpm for 5 minutes in independent systems.

Thereafter, the respective materials were charged into a mixing apparatus and were mixed at a speed of 250 rpm for 24 hours. At this time, the high-viscosity monomer and the low-viscosity monomer were sequentially introduced into the mixing apparatus.

The mixed composition was charged into a press-molding apparatus, and the temperature was raised from a starting temperature of 30° C. up to a final temperature of 129° C. at a heating temperature of about 8° C./hr. The press pressure was maintained at 25 MPa to 35 MPa, and the molding was carried out at the final temperature for 12 hours.

The press-molded block composition was simultaneously cured under a first wavelength range of 405 nm and a second wavelength range of 385 nm.

The cured block composition was removed from the mold to obtain a final product.

TABLE 1

| Component | | Example 1 | Example 2 | Example 3 | Example 4 | Example 5 |
|---|---|---|---|---|---|---|
| Base resin (A) | a1 | 4.4 | 4.5 | 4.5 | 4.5 | 4.5 |
| Inorganic filler (B) | b1 | 67.981 | 67.981 | 67.976 | 67.967 | 67.96 |
| | b2 | 5 | 5 | 5 | 5 | 5 |
| | b3 | 0.009 | 0.008 | 0.008 | 0.008 | 0.008 |
| Monomer (C) | c1 | 3.5 | 3.5 | 3.5 | 3.5 | 3.5 |
| | c2 | 5.106 | 5 | 5 | 5 | 5 |
| | c3 | 6 | 6 | 6 | 6 | 6 |
| | c4 | 6.5 | 6.5 | 6.5 | 6.5 | 6.5 |
| Photoinitiator (D) | d1 | 0.65 | 0.7 | 0.7 | 0.7 | 0.7 |
| | d2 | 0.35 | 0.3 | 0.3 | 0.3 | 0.3 |
| Polymerization | e1 | 0.5 | 0.5 | 0.5 | 0.5 | 0.5 |
| retarder (E) | | | | | | |
| Pigment (F) | f1 | 0.005 | 0.003 | 0.005 | 0.009 | 0.01 |
| | f2 | 0.003 | 0.007 | 0.01 | 0.015 | 0.021 |
| | f3 | 0.0003 | 0.001 | 0.001 | 0.001 | 0.001 |

Photographs of the surface colors of the resin blocks of Examples 1 to 5 are shown in the FIGURE From left to right, the resin blocks correspond to Example 1 (B1), Example 2 (A1), Example 3 (A2), Example 4 (A3), and Example 5 (A3.5). The five resin blocks have respective colors falling within the same white series. The color of the rightmost resin block A3.5 has the darkest shade among the five white-series colors (a beige color), and the color of the leftmost resin block B1 has the brightest color (a light ivory color). From resin block B1 toward resin block A3.5, the color of the resin blocks gradually becomes darker in shade.

According to the method for manufacturing the dental resin block of an exemplary embodiment of the present disclosure, various color differences within a fine range may be exhibited by adjusting the compositional ratio. According to such a resin block, a customized color suitable for matching the natural color of a patient's tooth may be provided.

### INDUSTRIAL APPLICABILITY

When the method for manufacturing a dental resin block according to embodiments of the present application is used, a customized color suitable for matching the natural color of a patient's tooth may be realized, and accordingly the present disclosure is expected to have high industrial applicability in the dental field.

1. A method for manufacturing a dental resin block, the method comprising:
preparing a block composition comprising a base resin, an inorganic filler, and a monomer;
mixing the block composition;
press-molding the mixed block composition; and
curing the press-molded block composition under a first wavelength range of 395 nm to 550 nm and a second wavelength range of 200 nm to 393 nm.

2. The method for manufacturing a dental resin block of claim 1, wherein the curing of the press-molded block composition is performed for 5 hours to 24 hours.

3. The method for manufacturing a dental resin block of claim 1, wherein the preparing of the block composition comprising the base resin, the inorganic filler, and the monomer comprises separately dispersing each of the base resin, the inorganic filler, and the monomer in independent systems.

4. The method for manufacturing a dental resin block of claim 3, wherein the separately dispersing of each of the base resin, the inorganic filler, and the monomer is performed under conditions of 1,000 rpm to 5,000 rpm for 1 minute to 30 minutes.

5. The method for manufacturing a dental resin block of claim 1,
wherein the monomer comprises a high-viscosity monomer and a low-viscosity monomer having viscosities different from each other, and
wherein the mixing of the block composition comprises sequentially introducing the high-viscosity monomer and the low-viscosity monomer into a mixer.

6. The method for manufacturing a dental resin block of claim 5, wherein the high-viscosity monomer has a dynamic viscosity at 25° C. of 3,000 cPs or more, and the low-viscosity monomer has a dynamic viscosity at 25° C. of 2,500 cPs or less.

7. The method for manufacturing a dental resin block of claim 1, wherein the mixing of the block composition is performed under mixing conditions of 100 rpm to 600 rpm for 1 hour to 48 hours.

8. The method for manufacturing a dental resin block of claim 1, wherein the press-molding of the mixed block composition comprises increasing temperature from a starting temperature of 25° C. to 35° C. up to a final temperature of 100° C. to 130° C. at a heating rate of 2° C./hr to 12° C./hr.

9. The method for manufacturing a dental resin block of claim 1, wherein the press-molding of the mixed block composition is performed under a pressure of 5 MPa to 50 MPa for 5 hours to 48 hours.

10. The method for manufacturing a dental resin block of claim 1, wherein the base resin comprises a polyaryletherketone resin.

11. The method for manufacturing a dental resin block of claim 1, wherein the inorganic filler has a average particle diameter (D50) of 0.1 μm to 50 μm.

12. The method for manufacturing a dental resin block of claim 1, wherein the inorganic filler comprises silica, aluminum silicate, alumina, calcium fluoride, strontium fluoride, calcium carbonate, kaolin, clay, mica, aluminum sulfate, calcium sulfate, barium sulfate, titanium oxide, calcium phosphate, hydroxyapatite, calcium hydroxide, strontium hydroxide, zeolite, titanium dioxide, zirconia or combinations thereof.

13. The method for manufacturing a dental resin block of claim 1, wherein the inorganic filler comprises an inorganic glass filler.

14. The method for manufacturing a dental resin block of claim 1, wherein an amount of the monomer is 100 parts by weight to 1,000 parts by weight, based on 100 parts by weight of the base resin.

15. A dental resin block manufactured by the method for manufacturing a dental resin block according to claim 1.
